(19) United States
(12) Plant Patent Application Publication
(10) Pub. No.: US 2008/0201809 P1
(43) Pub. Date: Aug. 21, 2008

(54) Grapevine (Sheegene-15)

(76) Inventor: Timothy P. Sheehan, Porterville, CA (US)

Correspondence Address: Eric C. Cole, Esq.;Baker, Manock & Jensen, Ste. 421, 5260 N. Palm Ave.,, Fresno, CA, 93704, US

(21) Appl. No.: 11/708,798

(22) Filed: Feb. 20, 2007

(51) Int. Cl. A01H 5/00

(52) U.S. Cl. PLT/207

(57) ABSTRACT

A new and distinct variety characterized by a green-colored, medium sized seedless grape that ripens during the fourth week of June, approximately two to three weeks earlier than the Perlette Seedless Grape (unpatented), when grown in the San Joaquin Valley of Central California. The berries of this new variety are produced on strong woody branches and are well adapted to commercial handling.

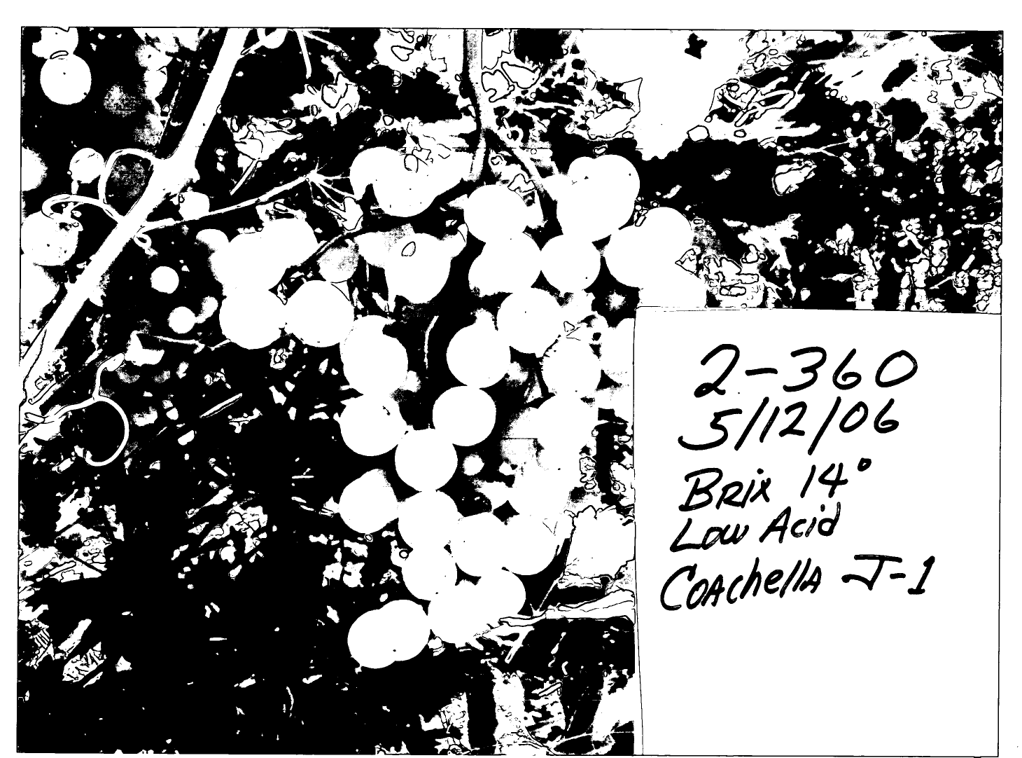

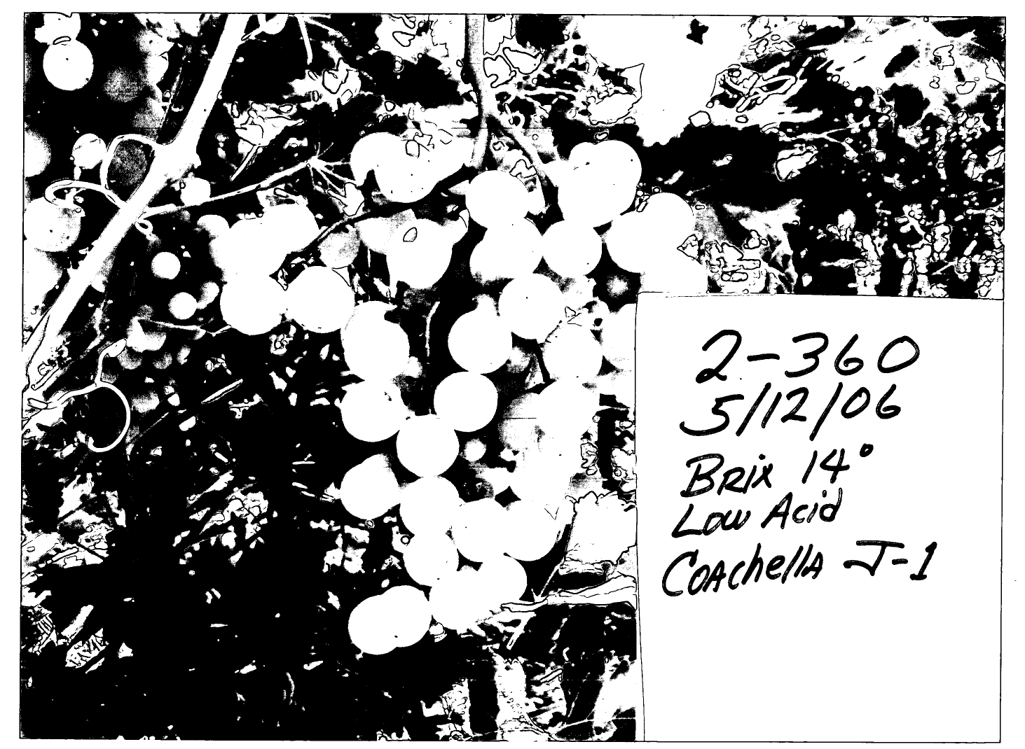

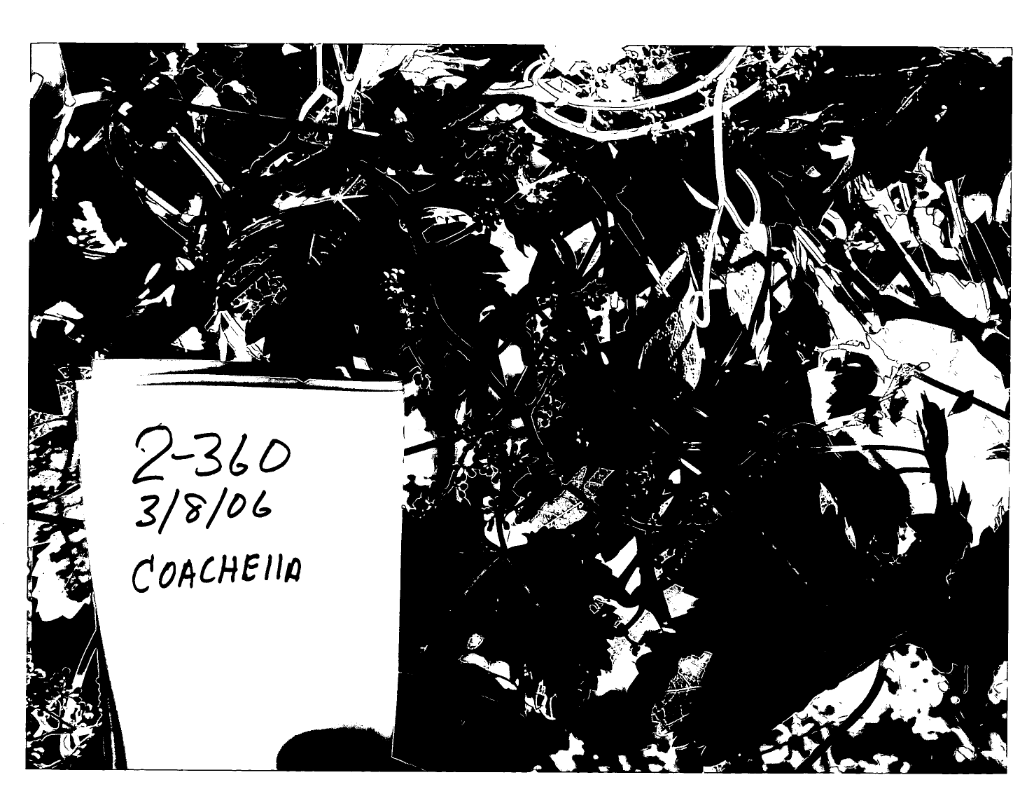

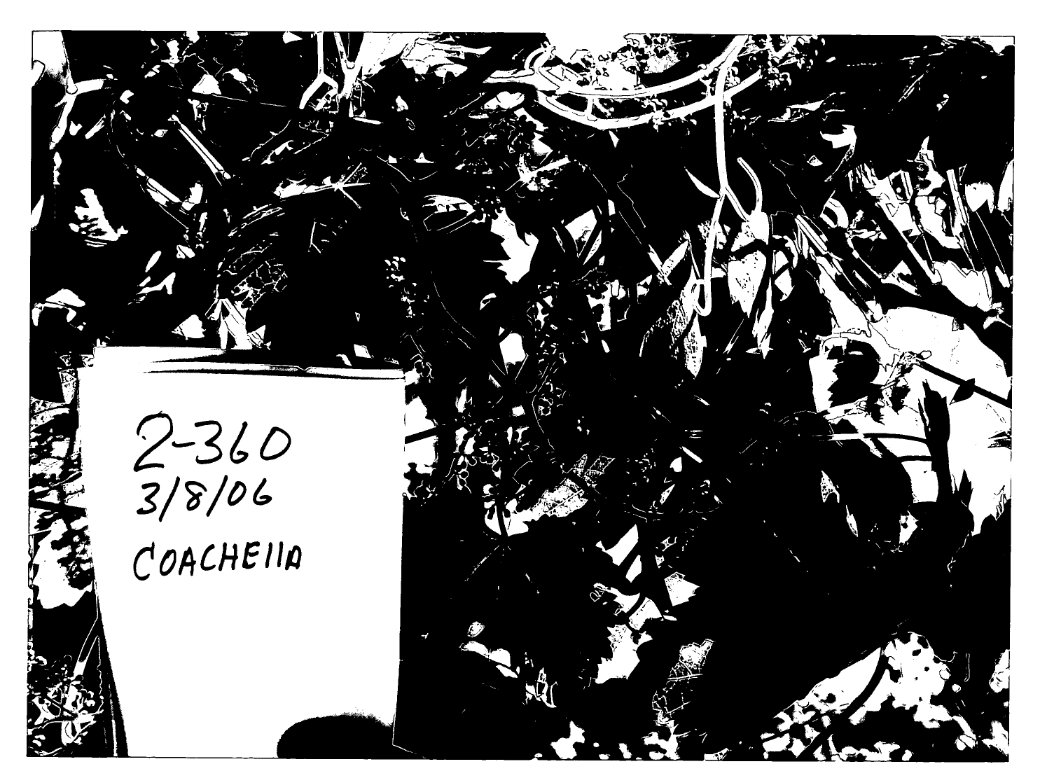

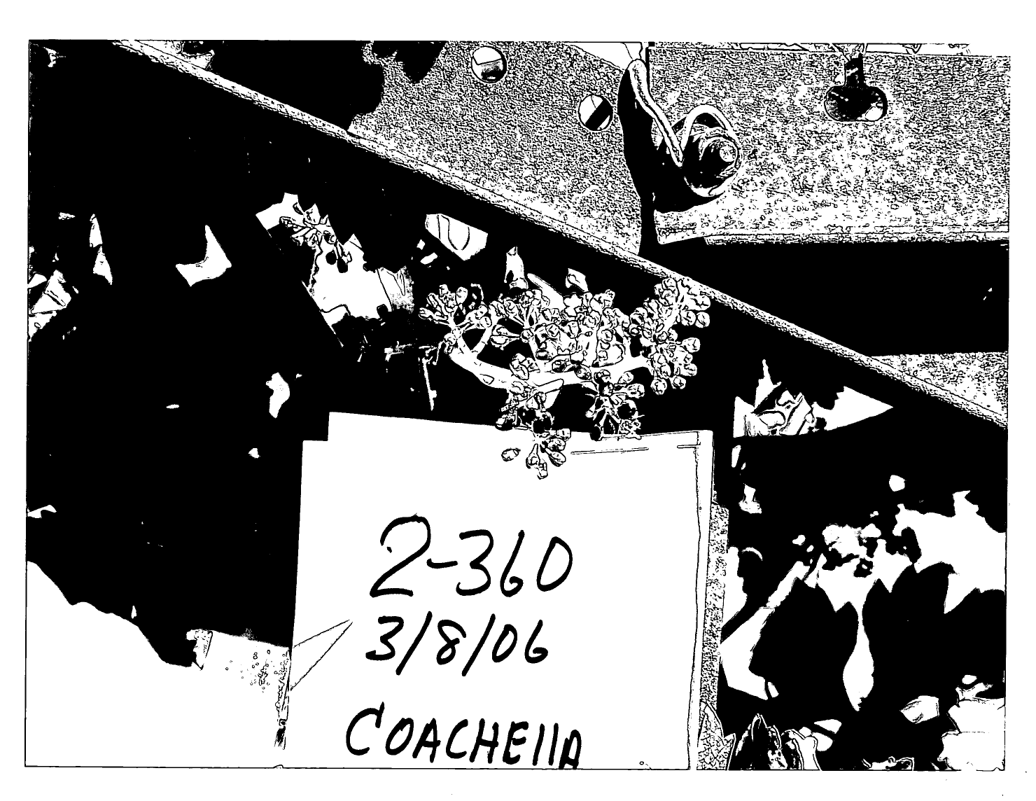

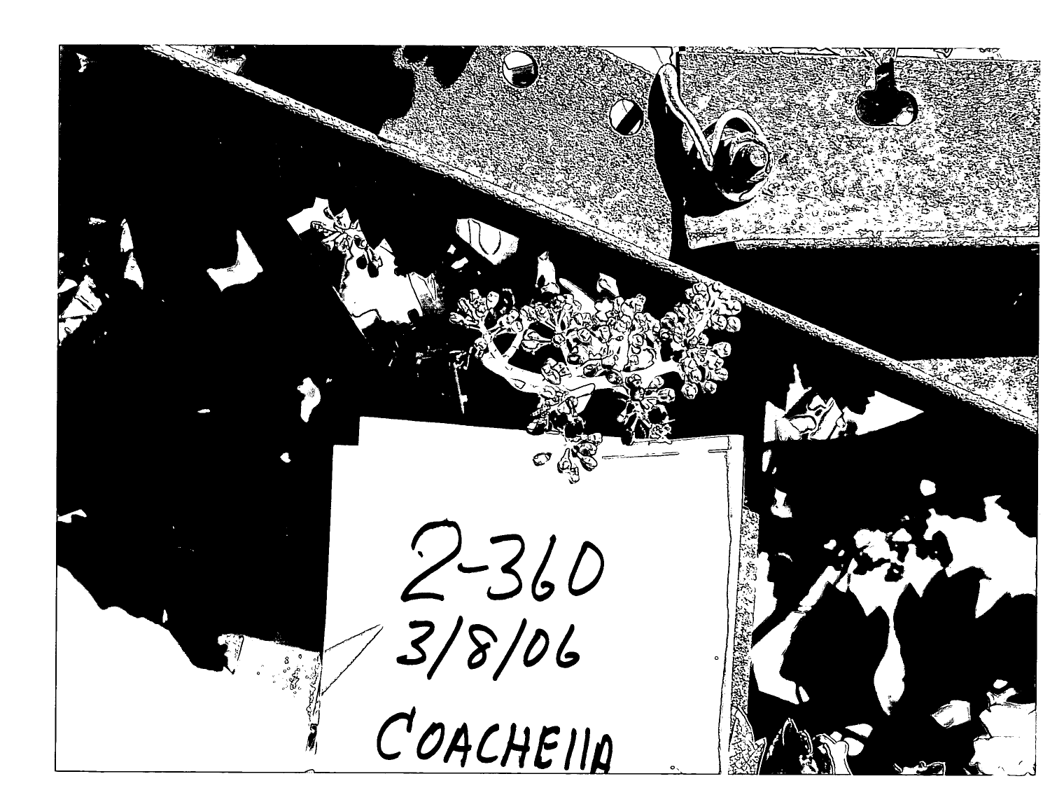

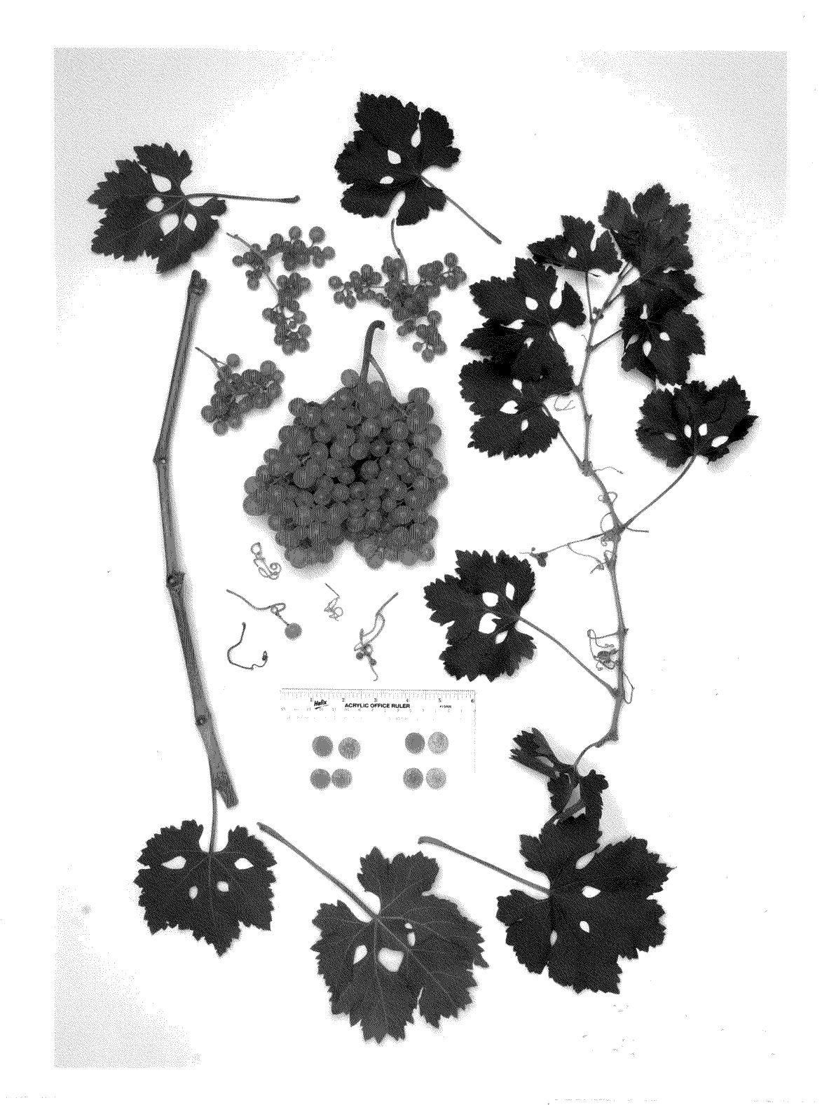

### CROSS REFERENCE TO RELATED APPLICATIONS

The claimed plant is not subject of a related application.

### STATEMENT OF ANY FEDERALLY-SPONSORED RESEARCH AND DEVELOPMENT

The claimed plant is not subject of Federally-sponsored research or development.

### LATIN NAME OF THE GENIUS AND SPECIES OF THE PLANT CLAIMED

The claimed plant relates to a new and distinct variety of Vitis vinifera to be known as ‘Sheegene-15’.

### VARIETY DENOMINATION

This invention relates to a new discovery and asexual reproduction of a new variety of Vitis vinifera. The new variety was first hybridized by Timothy P. Sheehan of Porterville, Calif. in Spring 2000; first flowering occurred in May 2003. The pollen parent is Brickeys Best (unpatented) and the seed parent is Red Globe (U.S. Plant Pat. No. 4,787). The new variety was asexually propagated in the dormant season of 2003/2004, bud grafted on Harmony, virus-free rootstock. Four vines were planted at that time in a Vitis vinifera variety block near Fowler, Calif. on the west side of Thompson Road, north of Adams Road. The green-colored seedless grapes produced by the new variety are medium in size and mature at least two to three weeks before Perlette Seedless (unpatented). The new variety has been shown to maintain its distinguishing characteristics through asexual propagation.

### BACKGROUND OF THE INVENTION

The present invention relates to a new and distinct variety of grape which will hereinafter be denominated as ‘Sheegene-15’ and more particularly as a grapevine which produces a green-colored seedless table grape that matures during the fourth week of June in the San Joaquin Valley of Central California. The new invention most closely resembles Perlette Seedless (unpatented) but it produces a medium size grape that is sweet, has very good flavor, and matures at least two to three weeks earlier.

### SUMMARY OF THE INVENTION

The ‘Sheegene-15’ grapevine is characterized by producing a medium size green seedless grape with very good flavor that is mature for harvesting and shipment in the fourth week of June in the San Joaquin Valley of Central California. The new variety can be compared to Perlette Seedless (unpatented) but the grapes mature at least two to three weeks earlier, among other distinguishing characteristics.

### BRIEF DESCRIPTION OF THE DRAWINGS

The accompanying drawing is a color photograph of the grapes produced by the subject variety; several leaves are displayed as well as a typical section of the vine. Across the top and bottom of the color photograph are leaves displaying the top and bottom surfaces of the leaves. The upper-middle section of the color photograph shows a major grape cluster. The lower-middle section of the color photograph shows four grapes cut in half longitudinally as well as horizontally displaying the flesh and shape. Along the left side of the color photograph is a section of mature cane displaying a leaf and nodes. Along the right side of the color photograph is an immature cane displaying several tendrils and leaves.

### DETAILED BOTANICAL DESCRIPTION

Referring more to the horticultural description of the new and distinct variety of grapevine, the following description has been observed under the ecological conditions prevailing at the origin vineyard that is located east of Fowler, Calif. in the San Joaquin Valley of Central California.

All major color descriptions are by reference to the Dictionary of Color by Maerz and Paul, First Edition, published in 1930. Common color names are also used in several instances.
Vine:
   Size.—Large for a four year old vine.
   Vigor.—Very good.
   Chilling requirements.—Normal for grapes in San Joaquin Valley of Central California.
   Productive capacity.—Very good.
   Regularity of bearing.—Regular.
Trunk:
   Size.—Medium to large, 28.21 cm (11-½ inches) in circumference 24 inches above graft.
   Surface texture.—Rough, shaggy.
   Inner bark color.—Pl.7 E9 Kaffa Flamered Brown.
   Mature cane color.—Pl.7 H7 Cedar +.
   Nodes.—Six on canes, 72.7 cm (28-⅝ inches) long.
   Length between nodes.—8.89 to 11.43 cm (3-½ to 4-½ inches).
   Lenticels — number.—0.
   Lenticels — size.—0.
   Shoots — length.—22 to 32 cm (8.66 to 12.50 inches).
   Shape — shape.—Droopy.
   Tendrils — number.—One at node.
   Tendrils — length.—20 to 33 mm (0.966 to 1.299 inches).
   Tendrils — location.—At nodes.
   Tendrils — form.—Bifurcate.
   Tendrils — texture.—Firm.
   Bud shape.—Convex.
   Bud size.—7 to 8 mm (0.28 to 0.21 inches) in length, 6 to 7 mm (0.24 to 0.25 inches) in width.
   Bud fruitfulness.—Very good.
Branches:
   Size.—Medium; 5.08 to 6.35 cm (2 to 2-½ inches) in circumference.
   Cordons.—83.82 cm (33 inches) from trunk at one side, 78.74 cm (31 inches) from trunk on other side.
   Surface texture.—Slightly rough.
   Color — one year or older wood.—Pl.6 H10 Cedar Wood, Tokay.
   Color — immature branches.—Pl.19 L6 Calliste gr.
Leaves:
   Size.—Medium.
   Density.—Dense.
   Average length — mature leaf.—11 cm (4.33 inches).
   Average width — mature leaf.—15.2 cm (5.98 inches).
   Form.—Pentagonal.
   Texture — upper surface.—Smooth.
   Texture — lower surface.—Slightly rough.
   Color — leaf vein.—Pl.17 I6 Viridine y.
   Leaf margin.—Slightly undulate.
   Glandular characteristics.—0.
   Petiole size.—Moderate.
   Petiole length.—12 to 13.5 cm (4.72 to 5.31 inches).
   Petiole thickness.—3 mm (0.12 inches).
   Petiole color.—Pl.17 I6 Viridine y.
   Stem glands.—0.
   Stipules — size.—Medium; 3 mm (0.12 inches) in width, 5 mm (0.04 inches) in length.
   Stipules — color.—Pl.20 K6 Piquant gr.; Tip — Pl.55 L5 Rubient.
   Lobes.—Four.
   Tooth size.—10 mm (0.39 inches) in width; 7 cm (0.28 inches) in length.
   Tooth — number.—Three large between lobes.
   Tooth — shape.—Convex.
Inflorescence:
   Size.—21.59 to 27.30 cm (8-½ to 10-¾ inches).
   Number borne per spur.—2 to 3.
   Number borne per vine.—25 to 27.
Flowers:
   Flower bud size.—Small; 1 to 2 mm.
   Flower bud surface.—Glabrous.
   Flower bud quantity.—Many.
   Date of bloom.—Apr. 22, 2007.
   Date of full bloom.—Apr. 27, 2007.
   Size of bloom.—4 to 5 mm (0.16 to 0.20 inches).
   Petal color.—Pl.19 K10 to Bun Color.
   Petal size.—1 to 2 mm (0.039 to 0.780).
   Pistil color.—Near Neva gr. Pl. 17 L8.
   Amount of pollen.—N/A.
   Date of first viable berries.—Apr. 30, 2007.
   Size.—Generally small 2 mm (0.078 inches).
Fruit:
   Seed numbers.—0.
   Capstem pedicel.—7 to 13 mm (0.28 to 0.51 inches).
   Berry weight.—2.84 grams.
   Juice color.—Clear.
   Average cluster length (not including stem).—13.86 cm (5-½ inches).
   Average cluster diameter.—9.50 cm (3-¾ inches).
   Average cluster weight.—341 grams to 12 oz (¾ lbs.).
   Compactness.—Compact.
   Cluster form.—Conical.
   Stem.—Generally 4.45 to 5.12 cm (1-¾ to 2-¼ inches).
   Stem caliper.—4 mm (0.16 inches).
   Berry size.—Small to medium.
   Berry form.—Ovate.
   Berry numbers.—120.
   Average longitudinal berry size.—16 to 33 mm (0.63 to 0.87 inches).
   Average transverse to longitudinal berry size.—16 to 20 mm (0.63 to 0.79 inches).
Skin:
   Skin thickness.—Medium.
   Texture.—Semi-tough.
   Tendency to crack.—None.
   Blush color.—None.
   Ground color.—Pl. 20 K6 Piquant gr.
   Pulp.—Clear.
   Lenticels.—None.
Flesh:
   Flesh color.—Pl.20 L6 Near Piquant gr.
   Juice production.—Very good.
   Flavor.—Very good.
   Aroma.—Very mild.
   Texture.—Firm.
   Ripening.—Even.
   Eating quality.—Very good.
   Use.—Fresh market.

Having thus described and illustrated our new variety of grapevine, we claim:

1. A new variety of grapevine to be known as ‘Sheegene-15’ and parts thereof, substantially as illustrated and described, characterized principally by its production of green-colored, medium sized seedless grapes, that mature earlier in the season of ripening than does the Perlette Seedless Grape (unpatented) which it closely resembles.
